(19) United States
(12) Patent Application Publication
(10) Pub. No.: US 2010/0057081 A1
(43) Pub. Date: Mar. 4, 2010

(54) Tissue Fusion Jaw Angle Improvement

(75) Inventor: D. Alan Hanna, Boulder, CO (US)

Correspondence Address: TYCO Healthcare Group LP;Attn: IP Legal, 5920 Longbow Drive, Mail Stop A36, Boulder, CO, 80301-3299, US

(73) Assignee: TYCO Healthcare Group LP

(21) Appl. No.: 12/200,154

(22) Filed: Aug. 28, 2008

(51) Int. Cl. A61B 18/04

(52) U.S. Cl. 606/51

(57) ABSTRACT

A bipolar forceps for sealing tissue includes an end effector assembly having opposing first and second jaw members having a proximal end and a distal end. The jaw members are moveable relative to one another from a first spaced apart position to a second position in which the jaw members cooperate to grasp tissue. Each of the jaw members includes an electrode having an electrically conductive tissue sealing surface. An electrical energy source may be connected to the tissue sealing surfaces so that the sealing surfaces can conduct energy to tissue. Each electrode may be pivotably connected to the respective jaw member to promote parallel closure of the sealing surfaces against tissue between the jaw members. Each electrode may be wedge-shaped such that the thickness of the electrode increases distally along a length thereof to promote parallel closure of the sealing surfaces against tissue between the jaw members.

### BACKGROUND

1. Background

The present disclosure relates to electrosurgical forceps for assuring uniform sealing of tissue when performing electrosurgical procedures. More particularly, the present disclosure relates to open, laparoscopic, or endoscopic bipolar forceps that improve the uniformity of current distribution through tissue and create a seal having a substantially uniform tissue thickness, by improving parallelism of the electrode faces of the bipolar forceps.

2. Technical Field

Forceps utilize mechanical action to constrict, grasp, dissect and/or clamp tissue. Electrosurgical forceps utilize both mechanical clamping action and electrical energy to effect hemostasis by heating the tissue and blood vessels. By controlling the intensity, frequency and duration of the electrosurgical energy applied through jaw members to the tissue, the surgeon can coagulate, cauterize and/or seal tissue.

In order to effect a proper seal with larger vessels or thick tissue, two predominant mechanical parameters must be accurately controlled—the pressure applied to the tissue and the gap distance between the electrodes. As can be appreciated, both of these parameters are affected by thickness of vessels or tissue. More particularly, accurate application of pressure is important for several reasons: to oppose the walls of the vessels; to reduce the tissue impedance to a low enough value that allows enough electrosurgical energy through the tissue; to overcome the forces of expansion during tissue heating; and to contribute to the end tissue thickness which is an indication of a good seal. It has been determined that a fused vessel wall is optimum between 0.001 and 0.006 inches. Below this range, the seal may shred or tear and above this range the lumens may not be properly or effectively sealed.

With respect to smaller vessels, the pressure applied to the tissue tends to become less relevant whereas the gap distance between the electrically conductive tissue sealing surfaces becomes more significant for effective sealing. In other words, the chances of two electrically conductive sealing surfaces touching during activation increases as the vessels become smaller.

Electrosurgical methods may be able to seal larger vessels using an appropriate electrosurgical power curve, coupled with an instrument capable of applying a large closure force to the vessel walls. It is thought that the process of coagulating small vessels is fundamentally different than electrosurgical tissue vessel sealing. For the purposes herein “coagulation” is defined as a process of desiccating tissue wherein the tissue cells are ruptured and dried and vessel sealing is defined as the process of liquefying the collagen in the tissue so that it reforms into a fused mass. Thus, coagulation of small vessels is sufficient to permanently close them. Larger vessels need to be sealed to assure permanent closure.

Numerous bipolar electrosurgical forceps have been proposed in the past for various surgical procedures. However, some of these designs may not provide uniformly reproducible pressure to the blood vessel and may result in an ineffective or non-uniform seal. Complicating matters further is the fact that a non-uniform pressure applied to a blood vessel creates varying tissue thickness along the length of the forceps. The result is varying pressure being applied, varying tissue thickness, and varying amount of electrosurgical energy passing through the tissue. All of these conditions reduce the effectiveness of the seal

### SUMMARY

A bipolar forceps for sealing tissue includes an end effector assembly having opposing first and second jaw members each having a proximal end and a distal end. The jaw members are moveable relative to one another from a first spaced apart position to a second position wherein the jaw members cooperate to grasp tissue.

Each of the jaw members includes an electrode having an electrically conductive tissue sealing surface. An electrical energy source may be connected to the tissue sealing surfaces so that the sealing surfaces can conduct energy to tissue. The tissue sealing surfaces may include at least one electrically non-conductive insulating member disposed thereon to prevent shorting between the sealing surfaces. The insulating member may also be an insulating ridge disposed along a length of the tissue sealing surface.

In one embodiment, one or both electrodes may be pivotably connected to a respective jaw member between the proximal and distal ends thereof to promote parallel closure of the respective electrically conductive tissue sealing surfaces against tissue disposed between the jaw members. The electrodes may be pivotably connected to the jaw members midway along the length of the jaw member.

In another embodiment, one or both of the electrodes may be wedge-shaped such that the thickness of the electrically conductive tissue sealing surface increases distally along a length thereof to promote parallel closure of the respective electrically conductive tissue sealing surfaces against tissue disposed between the jaw members.

### BRIEF DESCRIPTION OF THE DRAWINGS

Various embodiments of the present disclosure are described herein with reference to the drawings wherein:

FIG. 1 is a perspective view of an electrosurgical forceps in accordance with an embodiment of the present disclosure;

FIG. 2A is a side view of a pair jaw members including individually pivoting electrodes pivotally connected thereto in a first spaced apart position in accordance with the present disclosure;

FIG. 2B is a side view of the jaw members in a second grasping tissue position in accordance with the present disclosure;

FIG. 2C is a side view of the jaw members including an insulating member disposed on each tissue sealing surface of each electrode, the jaw members being disposed in the first position in accordance with another embodiment of the present disclosure;

FIG. 2D is a side view of the jaw members of FIG. 2C in the second position in accordance with the present disclosure;

FIG. 3A is a side view of the jaw members including a wedge shaped electrode disposed at a distal end of each jaw member in accordance with another embodiment of the present disclosure;

FIG. 3B is a side view of the jaw members of FIG. 3A shown in the second grasping position;

FIG. 3C is a side view of the jaw members including an insulating member disposed on each tissue sealing surface of each electrode, the jaw members being disposed in the first position in accordance with another embodiment the present disclosure;

FIG. 3D is a side view of the jaw members of FIG. 3C in the second position in accordance with the present disclosure;

FIG. 4A is a side view of jaw members having opposing electrodes thereof pivotally connected at the distal end and connected by a spring at the proximal end, in accordance with the present disclosure;

FIG. 4B is a side view of the jaw members of FIG. 4A in the second grasping position in accordance with the present disclosure;

FIG. 4C is a side view of the jaw members including an insulating member disposed on each tissue sealing surface of each electrode, in the first position in accordance with another embodiment of the present disclosure;

FIG. 4D is a side view of the jaw members of FIG. 4C in the second position in accordance with the present disclosure;

FIG. 5A is a side view of a pair of jaw members connected by a trapezoidal pivot mechanism including electrodes disposed at a distal end thereof and shown in an open, spaced apart position;

FIG. 5B is a side view of the jaw members of FIG. 5A having an insulating member disposed on each of the tissue sealing surfaces of the electrodes;

FIG. 5C is a side view of the jaw members of FIG. 5A shown in the second grasping position; and

FIG. 5D is a side view of the jaw members of FIG. 5B shown in the second position.

### DETAILED DESCRIPTION

Various embodiments of the present disclosure are described hereinbelow with reference to the accompanying drawings. Well-known functions or constructions are not described in detail to avoid obscuring the present disclosure in unnecessary detail. Those skilled in the art will understand that the present disclosure may be adapted for use with a laparoscopic instrument, an endoscopic instrument, or an open instrument; however, different electrical and mechanical connections and considerations may apply to each particular type of instrument. The novel aspects with respect to vessel and tissue sealing are generally consistent with respect to the open, laparoscopic, and endoscopic designs. In the drawings and in the description that follows, the term “proximal”, as is traditional, will refer to the end of the forceps that is closer to the user, while the term “distal” will refer to the end of the forceps that is further from the user.

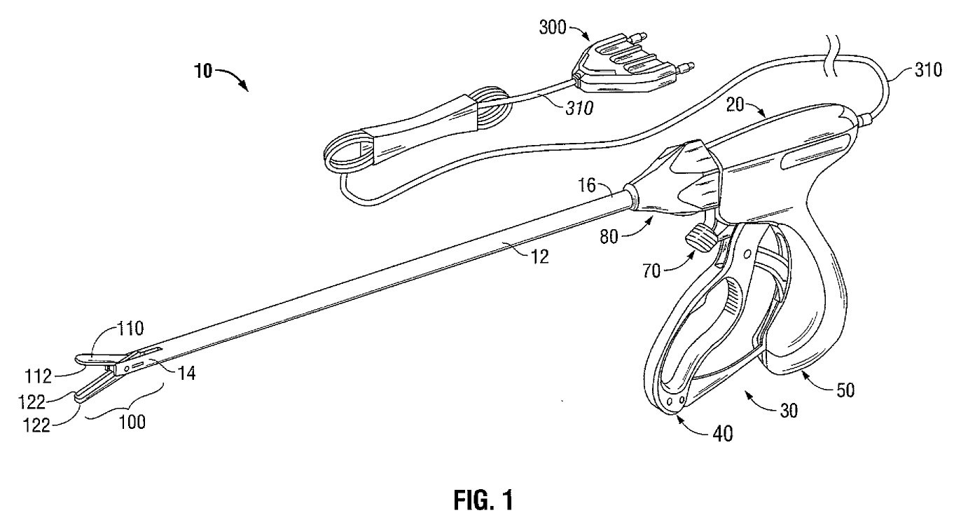

Referring now to FIG. 1, a bipolar electrosurgical forceps according to an embodiment of the present disclosure is shown including electrosurgical forceps 10 configured to support end effector assembly 100. Forceps 10 typically includes various conventional features (e.g., a housing 20, a handle assembly 30, a rotating assembly 80, a trigger assembly 70, etc.) that enable forceps 10 and end effector assembly 100 to mutually cooperate to grasp, seal and, if warranted, divide tissue. Forceps 10 generally includes housing 20 and handle assembly 30 that includes moveable handle 40 and handle 50 which is integral with housing 20. Handle 40 is moveable relative to handle 50 to actuate end effector assembly 100 to grasp and treat tissue. Forceps 10 also includes shaft 12 that has distal end 14 that mechanically engages end effector assembly 100 and proximal end 16 that mechanically en gages housing 20 proximate rotating assembly 80 disposed at the distal end of housing 20. Rotating assembly 80 is mechanically associated with shaft 12. Movement of rotating assembly 80 imparts similar rotational movements to shaft 64 which, in turn, rotates end effector assembly 100.

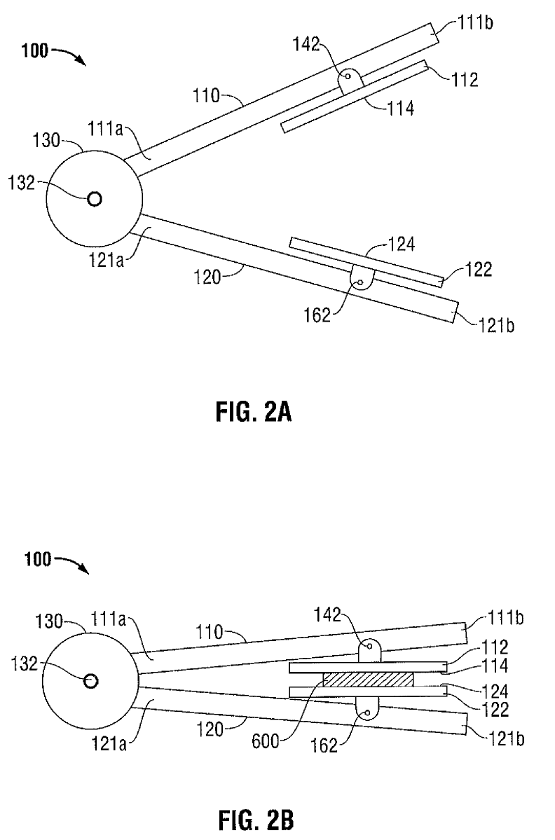

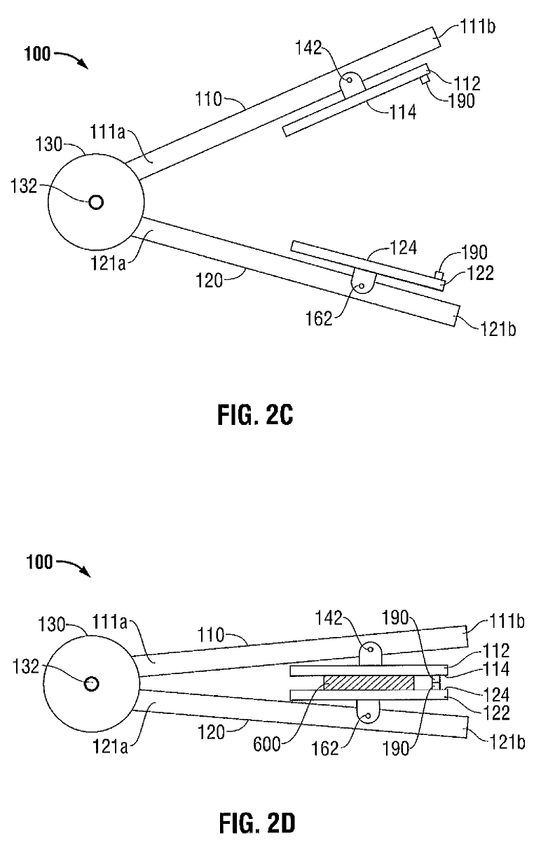

As explained in more detail below, with respect to FIGS. 2A-2D, end effector assembly 100 includes jaw members 110 and 120 having proximal ends 111a, 121a and distal ends 111b, 121b. Jaw members 110 and 120 are moveable from a first position wherein jaw members 110 and 120 are spaced relative to one another, to a second position wherein jaw members 110 and 120 are closed and cooperate to grasp tissue therebetween. Each jaw member 110, 120 includes respective electrodes 112 and 122 having an electrically conductive tissue sealing surface, 114 and 124, respectively, disposed on an inner-facing surface thereof. Electrically conductive tissue sealing surfaces 114 and 124 cooperate to seal tissue held therebetween upon the application of electrosurgical energy.

Referring now to FIGS. 2A-2D, end effector assembly 100 includes jaw members 110 and 120 connected at their respective proximal ends, 111a and 121a, via a suitable pivot mechanism 130. Jaw members 110 and 120 are rotatable about pivot pin 132 to effect grasping and sealing of tissue 600 (see FIG. 2B). Jaw members 110 and 120 include similar component features that cooperate to permit facile rotation about pivot pin 132. Other systems and methods for closing the jaws are possible and are within the purview of those skilled in the art. The jaw configuration may also be bilateral or unilateral.

Electrodes 112 and 122 are pivotally connected to the corresponding jaw members 110 and 120 via respective pivot mechanisms 142 and 162. As mentioned above, each electrode 112 and 122 has an electrically conductive tissue sealing surface 114, 124, respectively disposed thereon that are positioned to generally oppose one another, for grasping tissue therebetween.

As shown in FIG. 2B, as jaw members 110 and 120 are moved about pivot mechanism 130 relative to one another to grasp tissue 600, electrodes 112 and 122 tilt about respective pivots 142 and 162 such that electrically conductive tissue sealing surfaces 114 and 124 mutually cooperate in a substantially parallel manner to engage tissue. By assuring that the sealing surfaces 114 and 124 grasp tissue in a substantially parallel manner, the tissue thickness between electrodes 112 and 122 remains substantially uniform along the length of the sealing surfaces 114 and 124. This allows the surgeon to selectively apply a uniform closure pressure and a uniform amount of electrosurgical energy to tissue 600 between electrodes 112 and 122.

As shown in FIGS. 2C-2D, a pair of non-conductive insulating members 190 are disposed on electrically conductive tissue sealing surfaces 114 and/or 124 to prevent unintended shorting between the two electrically conductive tissue sealing surfaces 114 and 124. Insulating members 190 may also be used to maintain an effective gap distance between sealing surfaces 114 and 124 to promote tissue sealing, e.g. about 0.001 inches to about 0.006 inches. Insulating member 190 may also be configured as an insulating ridge disposed along a length of electrically conductive tissue sealing surface 114 or 124.

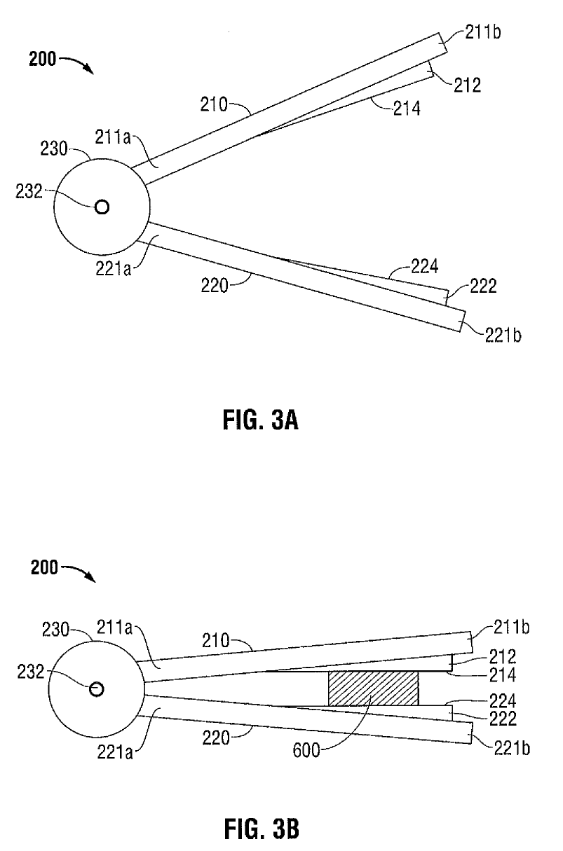

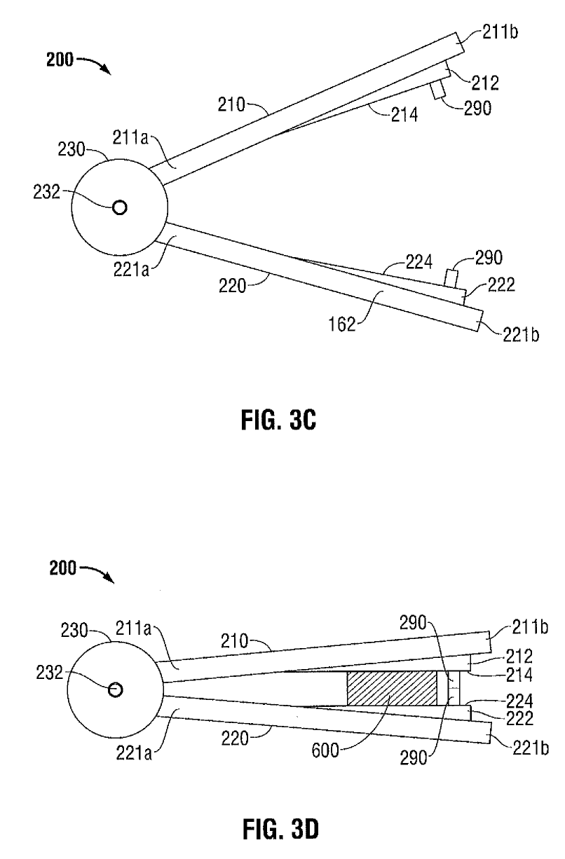

Referring now to FIGS. 3A-3D, in another embodiment, end effector assembly 200 includes jaw members 210 and 220 that are connected at their respective proximal ends, 211a and 221a, by a suitable pivot mechanism 230 and rotatable about pivot pin 232. The electrodes 212 and 222 are configured to be wedge-shaped, such that the thickness of electrodes 212 and 222 increases distally along a length thereof. Any suitable angle may be incorporated into the electrode to form the wedge-shape.

As shown in FIG. 3B, the wedge-shaped configuration of the electrodes 212 and 222 promotes parallel closure of respective electrically conductive tissue sealing surfaces 214 and 224 against tissue 600 disposed between jaw members 210 and 220. As the jaw members 210 and 220 move from the first position, as shown in FIGS. 3A and 3C, to the second position, as shown in FIGS. 3B and 3D, tissue 600 is squeezed toward the distal ends 211b and 221b of jaw members 210 and 220, respectively. At the same time, the wedged-shaped electrodes 212 and 222 squeeze tissue 600 toward the proximal ends 211a and 221a of jaw members 210 and 220, until tissue sealing surfaces 214 and 224 become parallel. Substantially parallel tissue sealing surfaces 214 and 224, as shown in FIGS. 3B and 3D, ensure that tissue thickness between electrodes 212 and 222 remains substantially uniform along a length of sealing surfaces 214 and 224. This enables a surgeon to apply accurate closure pressure and a proper amount of electrosurgical energy in a uniform fashion to seal tissue 600.

FIGS. 3C-3D show a pair of non-conductive insulating members 290 are disposed on the electrically conductive tissue sealing surfaces 214 and/or 224 to prevent unintended shorting between the two tissue sealing surfaces 214 and 224. Insulating members 290 may also be used to maintain an effective gap distance between sealing surfaces 214 and 224 to promote tissue sealing, e.g., about 0.001 inches to about 0.006 inches. Insulating members 290 may also be configured as insulating ridges disposed along a length of electrically conductive tissue sealing surface 214 and 224.

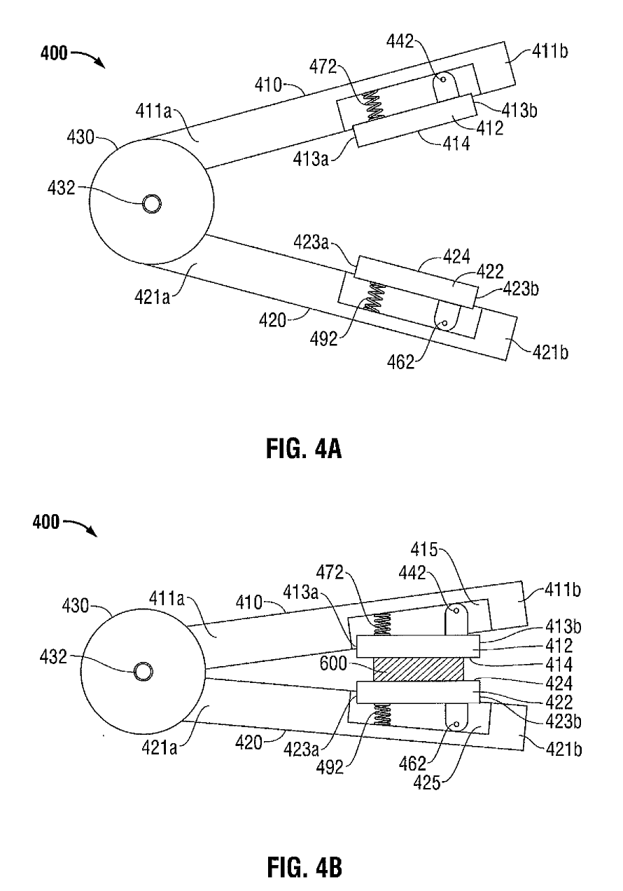

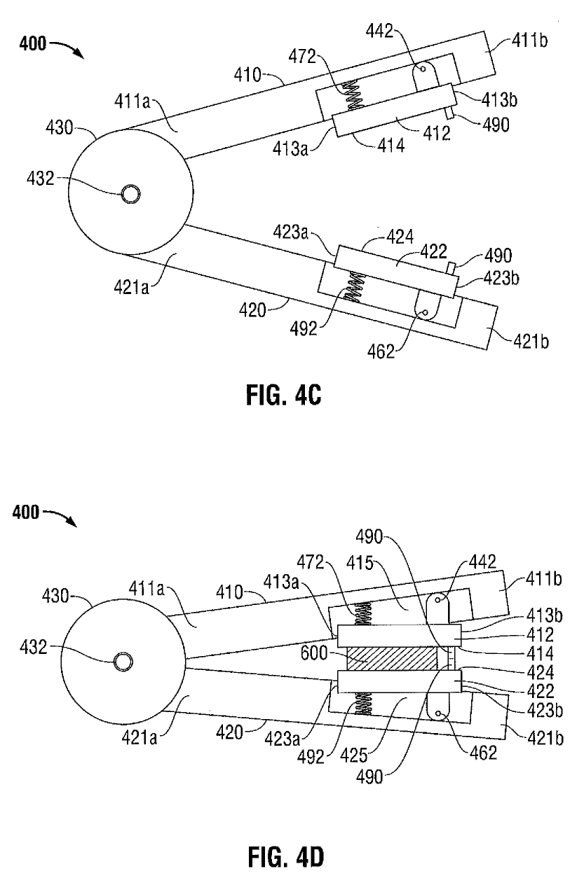

Referring now to FIG. 4A-4D, in another embodiment, end effector assembly 600 includes jaw members 410 and 420 pivotally connected to one another at proximal ends 411a and 421a via a suitable pivot mechanism 430 including pivot pin 432. A recess 415 and 425 (see FIG. 4D) may be defined within each jaw member 410 and 420, respectively. Electrodes 412 and 422 are disposed within each respective recess 415 and 425 and are pivotally connected to respective jaw members 410 and 420 at the distal ends 413b and 423b thereof. Alternatively, electrodes 412 and 422 may be connected to an inner facing surface of jaw members 410 and 420, respectively, similar to that shown in FIGS. 2A-2D. Each respective electrode 412 and 422 is also connected at the proximal end 413a and 423a thereof to jaw members 412 and 422, respectively, via resilient members 472 and 492, such that resilient members 472 and 492 bias each electrode 412 and 422 against tissue 600 disposed between jaw members 410 and 420. Resilient members 472 and 492 may be any compressible and/or flexible segment as is within the purview of those skilled in the art. In embodiments, resilient members 472 and 492 are springs. As shown in FIGS. 4B and 4D, as jaw members 410 and 420 are rotated about pivot pin 432 to the second position in order to grasp tissue 600 therebetween, electrodes 412 and 422 tilt about pivots 442 and 462 against springs 472 and 492 to compress tissue in a more parallel manner. As mentioned above in regards to previous embodiments, closing the electrodes and engaging tissue in a substantially parallel manner ensures that the tissue thickness between electrodes 412 and 422 remains substantially uniform along a length of sealing surfaces 414 and 424, thus allowing the surgeon to apply a uniform closure pressure and a uniform amount of electrosurgical energy to tissue 600 between electrodes 412 and 422.

FIGS. 4C and 4D show a pair of opposing insulating members 490 disposed on electrically conductive sealing surfaces 414 and 424 configured as insulating ridges disposed along a length of electrically conductive tissue sealing surface 414 and 424, as described above in relation to previous embodiments. Insulating members 490 prevent unintended shorting between the two tissue sealing surfaces 414 and 424. Insulating members 490 may also maintain an effective gap distance between sealing surfaces 414 and 424 to promote tissue sealing, e.g., about 0.001 inches to about 0.006 inches.

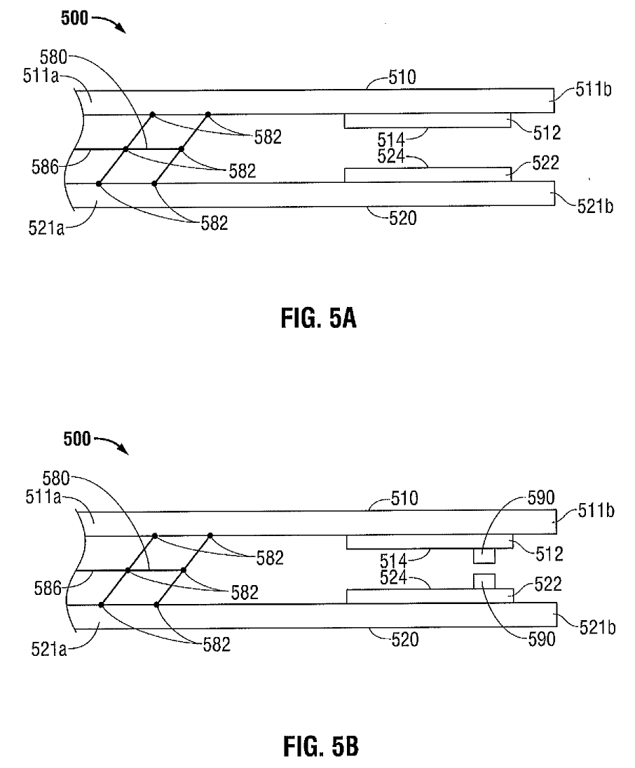

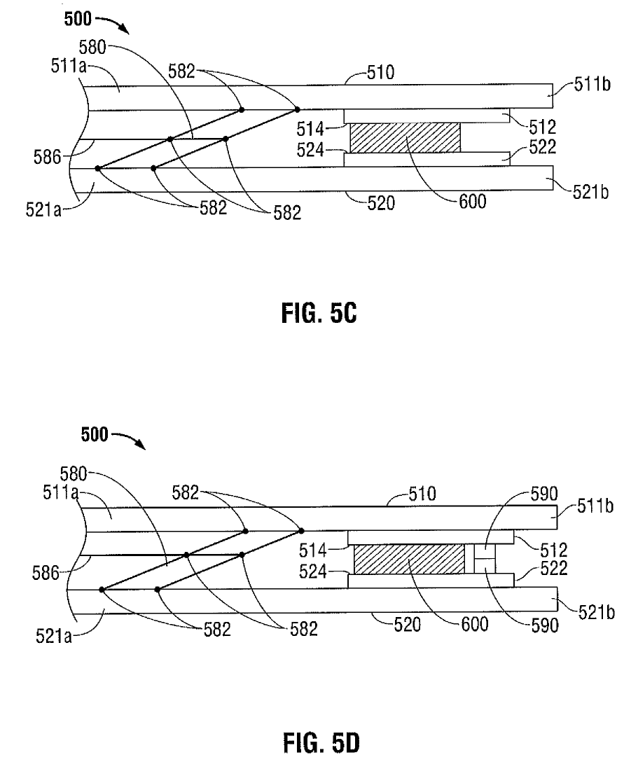

In yet another embodiment, as shown in FIGS. 5A-5D, jaw members 510 and 520 of end effector assembly 500 include electrodes 512 and 522, respectively, disposed on opposing surfaces thereon. Electrodes 512 and 522 include electrically conductive sealing surfaces 514 and 524, respectively. A trapezoidal pivot mechanism 580 operably connects jaw members 510 and 520 to one another via pivot connections 582. Pivot connections 584 connect an actuator rod 586 to trapezoidal pivot mechanism 580. When closure of jaw members 510 and 520 is required, e.g., by squeezing handle assembly 40, in order to grasp tissue therebetween, actuator rod 586 is advanced distally such that trapezoidal pivot mechanism 580 promotes a more parallel closure of jaw members 510 and 520, as shown in FIGS. 5C-5D. This results in parallel closure of tissue sealing surfaces 514 and 524, which ensures that tissue thickness between electrodes 512 and 522 remains substantially uniform along a length of sealing surfaces 514 and 524. The surgeon can selectively apply a uniform closure pressure and a uniform amount of electrosurgical energy to tissue 600 between electrodes 512 and 522.

As shown in FIGS. 5B and 5D, non-conductive insulating members 590 may also be disposed on electrically conductive tissue sealing surfaces 514 and 524 to prevent unintended shorting between the two electrically conductive tissue sealing surfaces 514 and 526. Insulating members 590 may also maintain an effective gap distance between sealing surfaces 514 and 524 to promote tissue sealing, e.g., about 0.001 inches to about 0.006 inches.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A bipolar forceps, comprising:
an end effector assembly including opposing first and second jaw members having proximal and distal ends and selectively moveable relative to one another from a first spaced apart position to a second position wherein the jaw members cooperate to grasp tissue therebetween, each of the jaw members including an electrode having an electrically conductive tissue sealing surface adapted to connect to an electrical energy source such that the electrically conductive tissue sealing surfaces are capable of conducting energy to tissue disposed therebetween,
wherein at least one of the electrodes is pivotably connected to a respective jaw member between the proximal and distal ends thereof to promote parallel closure of the electrically conductive tissue sealing surfaces against tissue disposed between the jaw members.

2. The bipolar forceps of claim 1, wherein both electrodes are pivotably connected to respective jaw members to promote parallel closure of the respective electrically conductive tissue sealing surfaces against tissue disposed between the jaw members.

3. The bipolar forceps of claim 1, wherein at least one of the electrically conductive tissue sealing surfaces includes at least one insulating member disposed along a length thereof to prevent unintended shorting between the two electrically conductive tissue sealing surfaces when the forceps is disposed in the second position.

4. The bipolar forceps of claim 3, wherein the at least one insulating member is configured as an insulating ridge disposed along a length of electrically conductive tissue sealing surface to prevent unintended shorting between the two electrically conductive tissue sealing surfaces when the forceps is disposed in the second position.

5. The bipolar forceps of claim 1, wherein the at least one electrode is pivotably connected to the jaw member midway along the length of the jaw member between the proximal and distal ends thereof.

6. The bipolar forceps of claim 1, wherein the at least one electrode is pivotably connected to the jaw member midway along the length of the electrode.

7. A bipolar forceps, comprising:
an end effector assembly including opposing first and second jaw members configured for selective movement relative to one another from a first spaced apart position to a second position wherein the jaw members cooperate to grasp tissue therebetween, each of the jaw members including an electrode having an electrically conductive tissue contacting surface adapted to connect to an electrical energy source such that the electrically conductive tissue sealing surfaces are capable of conducting energy to tissue disposed therebetween,
wherein at least one of the electrodes is wedge-shaped such that the thickness of the at least one electrode increases distally along a length thereof to promote parallel closure of the respective electrically conductive tissue sealing surfaces against tissue disposed between the jaw members.

8. The bipolar forceps of claim 7, wherein at least one of the electrically conductive tissue sealing surfaces includes at least one insulating member disposed along a length thereof to prevent unintended shorting between the two electrically conductive tissue sealing surfaces when disposed in the second position.

9. The bipolar forceps of claim 7, wherein at least one of the jaw members includes at least one insulating member disposed along a length thereof to prevent unintended shorting between the two electrically conductive tissue sealing surfaces when disposed in the second position.
